(19) United States
(12) Patent Application Publication
(10) Pub. No.: US 2025/0163018 A1
(43) Pub. Date: May 22, 2025

(54) KEY AMINO ACID BASED ON PROTEIN-DRUG INTERACTIONS INDUCED NEW CRYSTAL FORMS OF TRELAGLIPTIN AND PREPARATION METHODS THEREOF

(71) Applicant: GUANGDONG UNIVERSITY OF TECHNOLOGY, Guangzhou (CN)

(72) Inventors: Kailin XU, Guangzhou (CN); Jianping WANG, Guangzhou (CN); Jierui HUO, Guangzhou (CN); Jie BAI, Guangzhou (CN); Suqing ZHAO, Guangzhou (CN); Xinyu LIU, Guangzhou (CN); Liyin LIANG, Guangzhou (CN); Shujin LIAO, Guangzhou (CN)

(73) Assignee: GUANGDONG UNIVERSITY OF TECHNOLOGY, Guangzhou (CN)

(21) Appl. No.: 19/021,272

(22) Filed: Jan. 15, 2025

(30) Foreign Application Priority Data
Dec. 17, 2024 (CN) 202411857451.7

(51) Int. Cl. C07D 401/04

(52) U.S. Cl. CPC C07D 401/04

(57) ABSTRACT

The present disclosure discloses a key amino acid-induced new crystal forms of trelagliptin based on protein-drug interaction structure mining and preparation method thereof; the technical solution is to introduce key amino acids into the solvent crystallization system of trelagliptin to induce five new crystal forms of trelagliptin; the preparation method does not require the use of a large amount of organic solvent, and has the characteristics of simplicity, precision and high efficiency, with a strong ability of crystal form regulation, and energy saving, environmental protection, safety, and easy to realize green industrial production.

### CROSS-REFERENCE TO RELATED APPLICATIONS

This application claims priority to Chinese Patent Application No. 202411857451.7 with a filing date of Dec. 17, 2024. The content of the aforementioned application, including any intervening amendments thereto, is incorporated herein by reference.

### TECHNICAL FIELD

The present disclosure belongs to the technical field of drug preparation, in particular to a method for adjusting the drug crystal form of trelagliptin using key amino acids.

### BACKGROUND

The discovery and regulation of a new crystalline form of a drug is the core of drug crystallography research, which provides a basis for screening out advantageous crystalline forms with excellent physicochemical properties, significant therapeutic efficacy and high safety, and is of great value and practical significance to the research of drug crystalline forms. Differences in drug crystallization will affect the efficacy, safety, stability and formulation process of drugs.

The essence of crystallization is the process of molecular assembly, and intermolecular interactions (e.g., hydrophobic interactions, hydrogen bonding, van der Waals forces, electrostatic forces) will directly affect the assembly of molecules, which in turn affects the crystallization and crystal structure of substances. Previous studies have reported the interaction between trelagliptin and human serum albumin (HSA) and pepsin (PEP), which is the molecular interaction between the drug and the protein. Based on this interaction, key amino acids that have strong interactions with trelagliptin were excavated, and the differences in the interactions between the key amino acids and the drug molecules will change the assembly of the drug molecules, which will further affect the drug crystallization and the crystal structure of the drug. Currently, studies of key amino acid-induced drug crystallization based on protein-drug interaction mining have not been reported. Meanwhile, proteins are essentially formed by dehydration and condensation of amino acids to form peptides, and the peptide chains are coiled and folded to form the three-dimensional structure of proteins. Therefore, it is important to go deep into the protein to mine the key amino acids, and utilize the strong interactions between the key amino acids and the drug to study the effect of the key amino acids on the crystallization and crystalline form of the drug of trelagliptin, and to accurately induce a new crystalline form of the drug.

In this study, the key amino acids that play a strong interaction in the trelagliptin-human serum albumin (HSA) and trelagliptin-pepsin (PEP) interactions were selected as the research objects, and the key amino acids represented by glutamic acid (GLU), arginine (ARG), alanine (ALA), threonine (THR), and tyrosine (TYR), etc., and the formation of new crystal forms of trelagliptin induced by the key amino acids of protein-drug interaction and its preparation methods were discussed.

Trelagliptin (TRE), chemical name: 2-[[6-[(3R)-3-aminopiperidin-1-yl]-3-methyl-2,4-dioxopyrimidin-1-yl]methyl]-4-fluorobenzonitrile, with the chemical formula of C₁₈H₂₀FN₅O₂. Trelagliptin was researched by Takeda Pharmaceuticals in Japan and is a long-acting dipeptidyl peptidase-4 (DPP-4) inhibitor analog, and also a very good oral hypoglycemic drug used for the treatment of type II diabetes mellitus, with the structural formula shown below.

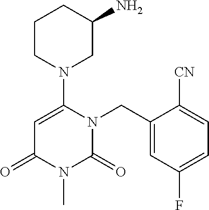 [SMILES: Cn1c(=O)cc(N2CCC[C@@H](N)C2)n(Cc2cc(F)ccc2C#N)c1=O]

TRE is known to be reported in various crystalline forms, such as PCT Patent Publication WO2014127735-A1 (title: Crystal form A of trelagliptin useful for e.g. preparing medicine for treating disease mediated by dipeptidyl peptidase IV, exhibits powder X-ray diffraction pattern, and has diffraction peak at specified angle) discloses a crystal form A of trelagliptin, which has an XRPD of The 2θ values are 5.7±0.2°, 11.4±0.2°, 12.5±0.2°, 16.8±0.2°, 17.1±0.2°, 19.4±0.2°, 19.9±0.2°, 20.5±0.2°, 22.5±0.2°, 22.9±0.2°, 29.1±0.2°. Next, Chinese patent publication CN105384724 (title: a crystalline form of a fluorine substituent and a method of preparation thereof) discloses trelagliptin crystalline form II and crystalline form IV and a method of preparation thereof; wherein, the 2θ values of the XRPD of the crystalline form II are 20.04°, 20.85°, and 21.86°, and the 2θ values of the XRPD patterns of the crystalline form IV are 16.28°, 20.03°, 22.30°, and 27.55°. For example, Chinese patent publication CN105693691 (title: New crystalline form of high purity trelagliptin and its preparation) discloses a method of preparing a new crystalline form of high purity trelagliptin, and the 2θ values of the XRPD pattern of this new crystalline form of trelagliptin are 4.8±0.2°, 9.6±0.2°, 18.4±0.2°, 18.9±0.2°. For example, Chinese patent publication CN104003975 (title: new solid form of trelagliptin and its preparation method and use) discloses five crystal forms of trelagliptin and its preparation method, including crystal form A, crystal form B, crystal form C, crystal form D, crystal form E and amorphous form. For example, Chinese Patent Publication CN115785066 (title: Trelagliptin new crystal form F and its preparation method) discloses a method for the preparation of trelagliptin new crystal form F. The new crystal form of trelagliptin was prepared by protein milling induction, which is a green and environmentally friendly preparation method.

However, most of the above methods have harsh conditions, and also do not employ safe and green amino acids as modulators to regulate the crystallization of trelagliptin. Based on the three-dimensional structure of trelagliptin-human serum albumin (HSA) and trelagliptin-pepsin (PEP) interactions, this patent has unearthed the key amino acids with strong interactions with the drug, innovatively introduces key amino acids into the trelagliptin solvent system, which induced the formation of various new crystalline shapes of trelagliptin and designed the method for its preparation. Glutamic acid (GLU), phenylalanine (PHE), arginine (ARG), threonine (THR) and tyrosine (TYR) were selected as the representative amino acids for the relevant experiments.

### SUMMARY OF THE INVENTION

A first object of the present disclosure is to provide a preparation method for introducing key amino acids into the trelagliptin solvent crystallization system, and obtaining a variety of new crystal forms of trelagliptin through the interaction between the key amino acids and trelagliptin.

A second object is to provide five new crystalline forms of trelagliptin.

For this purpose, a first technical solution provided by the present disclosure is as follows:

A method for preparing new crystalline forms of trelagliptin induced by key amino acids based on protein-drug interactions, in which key amino acids are introduced into a solvent crystallization system of trelagliptin to induce the production of a plurality of new crystalline forms of trelagliptin.

Further, the above-the method of preparing new crystalline forms of trelagliptin induced by key amino acids based on protein-drug interactions comprises, in turn, the following steps:
1) Slowly add solvent dropwise to the trelagliptin until the trelagliptin is just dissolved and saturated, and obtain a trelagliptin solution for spare use;
2) Add amino acids to deionized water or 20% hydrochloric acid solution until the amino acids are dissolved and saturated to obtain an amino acid solution;
3) Then use a rubber dropper to slowly drop the amino acid solution prepared in 2) into the trelagliptin solution prepared in 1) in sequence, and the whole process is carried out in a constant temperature water bath at 25° C.-50° C.;
4) After the addition is completed, seal with plastic wrap and tie 30-35 apertures at the seal, and place in a fume hood to wait for the precipitation of crystals, to obtain a variety of new crystalline forms of trelagliptin;
The ratio of the amount of matter of the amino acid and trelagliptin is 1:2˜1:30.

Further, the above-method of preparing a new crystalline form of trelagliptin induced by a key amino acid based on protein-drug interactions, the amino acid is one of glutamic acid, arginine, threonine, tyrosine, or any combination thereof; the solvent for dissolving trelagliptin is one of ethyl acetate, isopropanol, acetone, methanol, ethanol, and acetonitrile.

The second technical solution provided by the present disclosure is the key amino acid-induced new crystal forms of trelagliptin based on the structure mining of protein-drug interactions, the new crystal forms of trelagliptin being TRE-I, TRE-II, TRE-III, TRE-IV, TRE-V, and TRE-VI.

Further, the new crystalline form of trelagliptin induced by key amino acids based on the structure mining of protein-drug interactions, said new crystalline form of TRE-I has XRPD pattern 2θ values of 5.66°±0.2°, 11.35°±0.2°, 12.47°±0.2°, 17.08°±0.2°, 19.88°±0.2°, 22.48°±0.2°, 22.87°±0.2°, and 27.54°±0.2°.

Further, the new crystalline form of trelagliptin induced by key amino acids based on protein-drug interactions, said new crystalline form of TRE-II has XRPD pattern 2θ values of 8.07°±0.2°, 13.31°±0.2°, 13.64°±0.2°, 16.29°±0.2°, 16.79°±0.2°, 17.40°±0.2°, 19.40°±0.2°, 21.33°±0.2°, 22.83°±0.2°, 26.26°±0.2°, 26.57°±0.2°, 26.79°±0.2°, 27.49°±0.2°, 27.90°±0.2°, 33.33°±0.2°.

Further, the new crystalline form of trelagliptin induced by key amino acids based on protein-drug interactions, said new crystalline form of TRE-III has XRPD pattern 2θ values of 9.99°±0.2°, 13.29°±0.2°, 15.27°±0.2°, 22.62°±0.2°, 26.79°±0.2°, 27.90°±0.2°, 30.36°±0.2°, and 40.73°±0.2°.

Further, the new crystalline form of trelagliptin induced by key amino acids based on protein-drug interactions, said new crystalline form of TRE-IV has XRPD pattern 2θ values of 5.47°±0.2°, 11.02°±0.2°, 12.88°±0.2°, 16.59°±0.2°, 20.08°±0.2°, 20.91°±0.2°, 22.17°±0.2°, 23.21°±0.2°, 27.84°±0.2°, 33.55°±0.2°.

Further, the new crystalline form of trelagliptin induced by key amino acids based on protein-drug interactions, said new crystalline form of TRE-V has XRPD pattern 2θ values of 5.49°±0.2°, 11.04°±0.2°, 12.47°±0.2°, 12.86°±0.2°, 16.59°±0.2°, 20.06°±0.2°, 20.88°±0.2°, 22.19°±0.2°, 28.08°±0.2°, 28.98°±0.2°, 30.47°±0.2°, 33.54±0.2°.

Compared with the Prior Art, the Technical Solution Provided by the Present Disclosure has the Following Technical Advantages:

1, the technical solution provided by the present disclosure is firstly innovative in introducing key amino acids into the solvent crystallization system of trelagliptin, combining only a trace amount of organic solvent, and obtaining a variety of new crystalline forms of trelagliptin and the preparation method through the interaction of different solvent systems.

2, the amino acids in the technical solution provided by the present disclosure, as essential components of the human body, are necessary for the synthesis of all proteins in the human body, and are combined in the drug, which is not only non-toxic and harmful, but also in line with the human body necessary for growth. At the same time, it avoids the use of a large number of toxic and harmful organic solvents, and not only prepares a new crystal form of trelagliptin, but also the preparation process is accurate, environmentally friendly and safe, which is in line with the national concept of green production.

3, the technical solution provided by the present disclosure is based on the interaction of key amino acids-trelagliptin, and five new crystal forms have been obtained, which broadens the study of drug crystal forms, and provides certain guidance for the production of subsequent drug formulations.

### BRIEF DESCRIPTION OF THE DRAWINGS

FIG. 1 is an X-ray powder diffraction pattern of five new crystalline forms of trelagliptin induced by key amino acids based on structural mining of protein-drug interactions with the original crystalline form of trelagliptin;

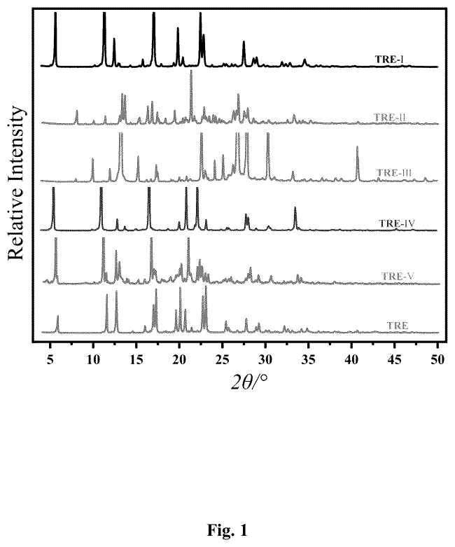

FIG. 2 is an X-ray powder diffraction pattern of the new crystalline type I prepared in Example 1;

FIG. 3 is an X-ray powder diffraction pattern of the new crystalline type II prepared in Example 2;

FIG. 4 is an X-ray powder diffraction pattern of the new crystalline type III prepared in Example 3;

FIG. 5 is an X-ray powder diffraction pattern of the new crystalline type IV prepared in Example 4;

FIG. 6 is an X-ray powder diffraction pattern of the new crystalline type V prepared in Example 5;

FIG. 7 is a microscopic observation diagram of the new crystalline type I prepared in Example 1;

FIG. 8 is a microscopic observation diagram of the new crystalline type II prepared in Example 2;

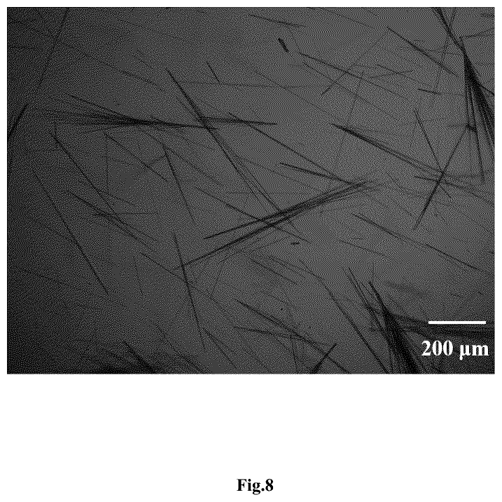

FIG. 9 is a microscopic observation diagram of the new crystalline type III prepared in Example 3;

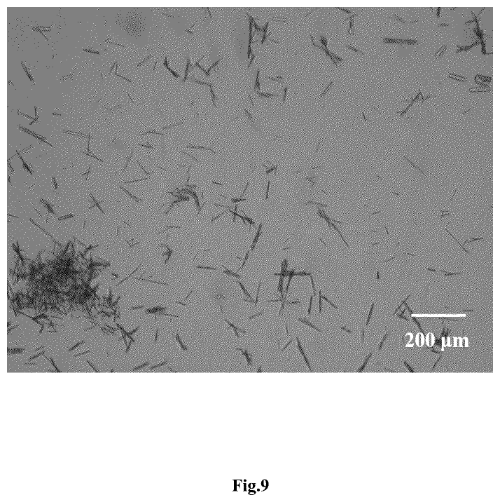

FIG. 10 is an observation diagram of Example 4 preparing a new crystalline type IV;

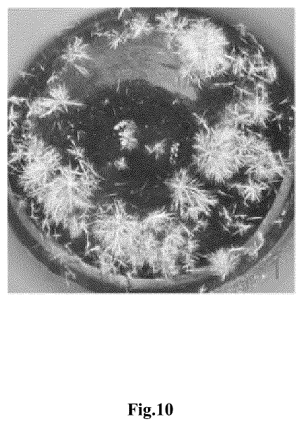

FIG. 11 is an observation diagram of Example 5 preparing a new crystalline type V.

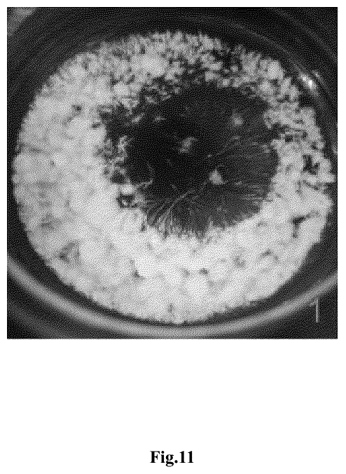

### DESCRIPTION OF THE EMBODIMENTS

A variety of new crystalline forms of trelagliptin induced by key amino acids in solvent systems and methods for their preparation are described below in connection with specific embodiments and the disclosure, but are not limited to these, and all related to this patent are protected.

### Example 1

Precisely weigh 100 mg of trelagliptin and add it to a 100 mL conical flask, then add 4 mL of methanol solvent to the conical flask and slowly add it dropwise until the trelagliptin is just dissolved and saturated, and keep it for use.

Take another 6.6 mg of threonine (Thr) and add to the conical flask, add 2 mL of deionized water to dissolve just to saturation.

Then use a rubber-tipped buret to slowly drop the amino acid solution prepared in step 2) into the solution prepared in step 1) in sequence, the whole process was carried out in a constant temperature water bath at 37° C.

After the addition was completed, the solution was sealed with plastic wrap and 30-35 apertures were tied at the seal, and placed in a fume hood to wait for the precipitation of crystals.

After the crystals were precipitated, an inverted microscope was used to observe the state of crystal growth and recorded. The crystals were separated from the solution by filtration, and the collected crystals were dried in a dry environment at T=30° C., and a portion of the resulting crystals were sent for XPRD measurement.

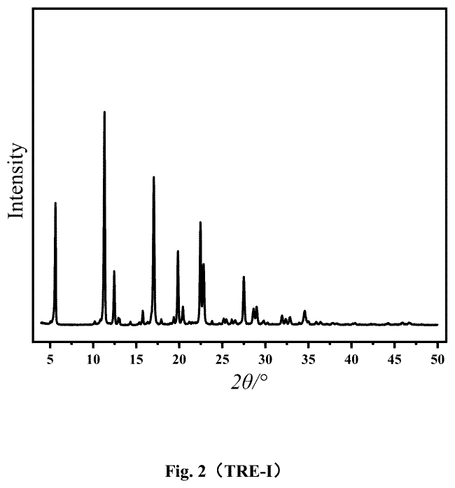

The XRPD patterns of crystalline trelagliptin were recorded under the following conditions: room temperature 25° C., relative humidity <60%; XPert PRO-type polycrystalline X-ray diffractometer (PANalytical, The Netherlands), Cu Kα radiation (λ=1.5406 Å), optical tube voltage 40 kV, tube current 40 mA, 2θ scanning range 4˜50°, step size 0.01313°, counting time 30 ms/step. The detection results are shown in FIG. 2. The 2θ values of the new crystalline TRE-I are 5.66°±0.2°, 11.35°±0.2°, 12.47°±0.2°, 17.08°±0.2°, 19.88°±0.2°, 22.48°±0.2°, 22.87°±0.2°, 27.54°±0.2°, compared with the existing crystalline forms, there are differences determined as new crystalline type.

### Example 2

Precisely weigh 150 mg of trelagliptin into a 100 mL conical flask, then add 6 mL of methanol solvent to the conical flask and slowly add dropwise until the trelagliptin is just dissolved and saturated, and set aside for use.

Take another 10 mg of tyrosine (Tyr) in a conical flask and add 3 mL of 20% hydrochloric acid solution to dissolve until just saturated.

The amino acid solution prepared in step 2) was then slowly dripped into the solution prepared in step 1) with a rubber-tipped burette, and the whole process was carried out at 50° C. in a thermostatic water bath.

After the addition was completed, the solution was sealed with plastic wrap and 30-35 apertures were tied at the sealing area, and placed in a fume hood to wait for the precipitation of crystals.

After the crystals were precipitated, an inverted microscope was used to observe the growth state of the crystals and recorded. The crystals were separated from the solution by filtration, and the collected crystals were dried in a dry environment at T=30° C., and a portion of the resulting crystals was sent for XPRD measurement.

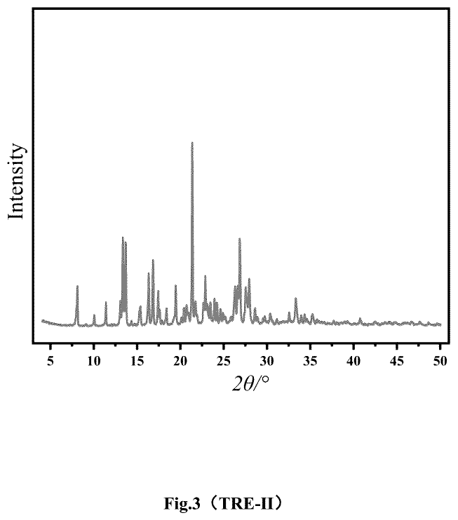

The X-ray powder diffraction patterns of crystalline trelagliptin were recorded under the following conditions: room temperature 25° C., relative humidity <60%; XPert PRO-type polycrystalline X-ray diffractometer (PANalytical, The Netherlands), Cu Kα radiation (λ=1.5406 Å), phototube voltage 40 kV, tube current 40 mA, 2θ scanning range 4˜50°, step size 0.01313°, counting time 30 ms/step, and the detection results are shown in FIG. 3. The 2θ values of the new crystalline TRE-II are 8.07°±0.2°, 13.31°±0.2°, 13.64°±0.2°, 16.29°±0.2°, 16.79°±0.2°, 17.40°±0.2°, 19.40°±0.2°, 21.33°±0.2°, 22.83°±0.2°, 26.26°±0.2°, 26.57°±0.2°, 26.79°±0.2°, 27.49°±0.2°, 27.90°±0.2°, 33.33°±0.2°, compared with the existing crystalline forms, there are differences determined as new crystalline type.

### Example 3

Precisely weigh 250 mg of trelagliptin into a 100 mL conical flask, then add 8 mL of acetonitrile or 6 mL of acetone solvent to the conical flask, and slowly add dropwise until the trelagliptin is dissolved and saturated, and set aside for use.

Take another 10 mg of tyrosine (Tyr) in a conical flask and add 3 mL of 20% hydrochloric acid solution to dissolve until just saturated.

The amino acid solution prepared in step 2) was then slowly dripped into the solution prepared in step 1) sequentially with a rubber-tipped burette, the whole process was carried out in a constant temperature water bath at 37° C.

After the addition was completed, the solution was sealed with plastic wrap and 30-35 apertures were tied at the seal, and placed in a fume hood to wait for the precipitation of crystals.

After the crystals were precipitated, an inverted microscope was used to observe the state of crystal growth and recorded. The crystals were separated from the solution by filtration, and the collected crystals were dried in a dry environment at T=30° C., and a portion of the resulting crystals was sent for XPRD measurement.

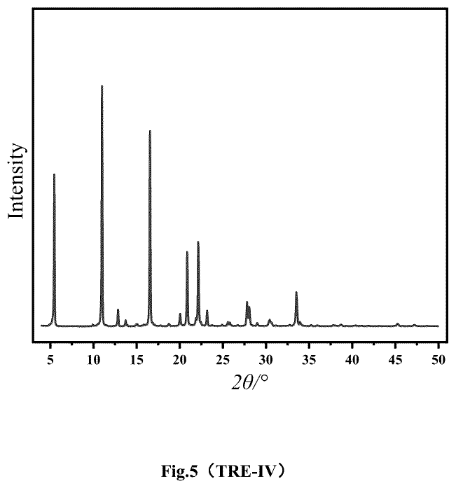

The X-ray powder diffraction patterns of crystalline trelagliptin were recorded under the following conditions: room temperature 25° C., relative humidity <60%; XPert PRO-type polycrystalline X-ray diffractometer (PANalytical, The Netherlands), Cu Kα radiation (λ=1.5406 Å), phototube voltage 40 kV, tube current 40 mA, 2θ scanning range 4˜50°, step size 0.01313°, counting time 30 ms/step, and the detection results are shown in FIG. 5. The 2θ values of the new crystalline TRE-III are 9.99°±0.2°, 13.29°±0.2°, 15.27°±0.2°, 22.62°±0.2°, 26.79°±0.2°, 27.90°±0.2°, 30.36°±0.2°, 40.73°±0.2°, compared with the existing crystalline forms, there are differences

### Example 4

Precisely weigh 100 mg of trelagliptin and add it to a 100 mL conical flask, then add 4 mL of ethyl acetate solvent to the conical flask and add it slowly dropwise until the trelagliptin is just dissolved and saturated, and set aside for use.

Then take 3.3 mg of arginine or 2.7 mg of glutamic acid or 3.3 mg of arginine mixed with 2.7 mg of glutamic acid system, either one of which was added to the conical flask, and 2 mL of deionized water was added to dissolve it until it was just dissolved and saturated.

The amino acid solution prepared in step 2) was then slowly dripped into the solution prepared in step 1) sequentially with a rubber-tipped buret, the whole process being carried out in a thermostatic water bath at 25° C.

After the addition was completed, the solution was sealed with plastic wrap and 30-35 apertures were tied at the seal, and placed in a fume hood to wait for the precipitation of crystals.

After the crystals were precipitated, an inverted microscope was used to observe the state of crystal growth and recorded. The crystals were separated from the solution by filtration, and the collected crystals were dried in a dry environment at T=30° C., and a portion of the resulting crystals were sent for XPRD measurement.

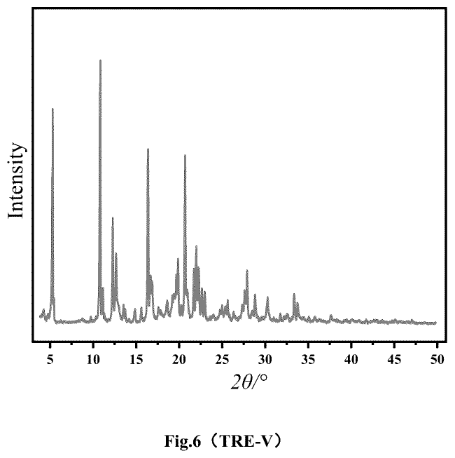

X-ray powder diffraction patterns of crystalline trelagliptin were recorded under the following conditions: room temperature 25° C., relative humidity <60%; XPert PRO-type polycrystalline X-ray diffractometer (PANalytical, The Netherlands), Cu Kα radiation (λ=1.5406 Å), phototube voltage 40 kV, tube current 40 mA, 2θ scanning range 4˜50°, step size 0.01313°, counting time 30 ms/step, and the detection results are shown in FIG. 6. The 2θ values of the new crystalline TRE-IV are 5.47°±0.2°, 11.02°±0.2°, 12.88°±0.2°, 16.59°±0.2°, 20.08°±0.2°, 20.91°±0.2°, 22.17°±0.2°, 23.21°±0.2°, 27.84°±0.2°, 33.55°±0.2°, compared with the existing crystalline forms, there are differences determined as new crystalline type.

### Example 5

Precisely weigh 100 mg of trelagliptin and add it to a 100 mL conical flask, then add 4.5 mL of isopropanol solvent to the conical flask and add it slowly dropwise until the trelagliptin is just dissolved and saturated, and set aside for use.

Then take 3.3 mg of arginine or 2.7 mg of glutamic acid or 3.3 mg of arginine mixed with 2.7 mg of glutamic acid system, either one of which was added to the conical flask, and 2 mL of deionized water was added to dissolve it until it was just dissolved and saturated.

The amino acid solution prepared in step 2) was then slowly dripped into the solution prepared in step 1) sequentially with a rubber-tipped buret, the whole process being carried out in a constant temperature water bath at 40° C.

After the addition was completed, the solution was sealed with plastic wrap and 30-35 apertures were tied at the seal, and placed in a fume hood to wait for the precipitation of crystals.

After the crystals were precipitated, an inverted microscope was used to observe the state of crystal growth and recorded. The crystals were separated from the solution by filtration, and the collected crystals were dried in a dry environment at T=30° C., and a portion of the resulting crystals were sent for XPRD measurement.

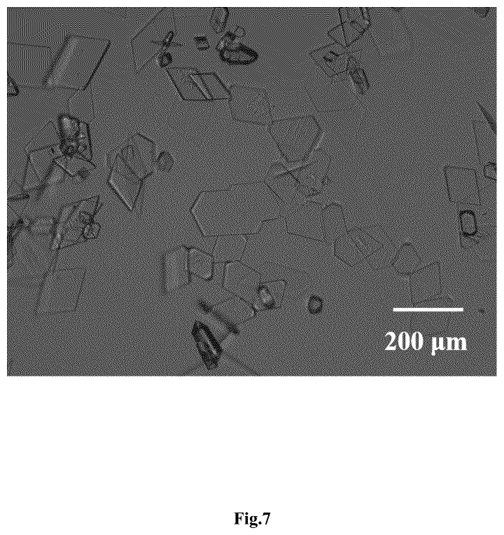

X-ray powder diffraction patterns of crystalline trelagliptin were recorded under the following conditions: room temperature 25° C., relative humidity <60%; XPert PRO-type polycrystalline X-ray diffractometer (PANalytical, The Netherlands), Cu Kα radiation (λ=1.5406 Å), phototube voltage 40 kV, tube current 40 mA, 2θ scanning range 4˜50°, step size 0.01313°, counting time 30 ms/step, and the detection results are shown in FIG. 7. The 2θ values of the new crystalline TRE-V are 5.49°±0.2°, 11.04°±0.2°, 12.47°±0.2°, 12.86°±0.2°, 16.59°±0.2°, 20.06°±0.2°, 20.88°±0.2°, 22.19°±0.2°, 28.08°±0.2°, 28.98°±0.2°, 30.47°±0.2°, 33.54±0.2°, compared with the existing crystalline forms, the presence of differences identified as new crystalline forms.

### Example 6

(1) Precisely weigh 200 mg of trelagliptin into a 100 mL conical flask, then add 6 mL of ethanol solvent to the conical flask, slowly add dropwise until the trelagliptin is just saturated with dissolution, and set aside for use.

(2) Take 10 mg of tyrosine (Tyr) in a conical flask and add 3 mL of 20% hydrochloric acid solution to dissolve until just saturated.

(3) The amino acid solution prepared in step 2) was then slowly dripped into the solution prepared in step 1) with a rubber-tipped burette, and the whole process was carried out in a constant temperature water bath at 40° C.

4) After the addition was completed, the solution was sealed with plastic wrap and 30-35 apertures were tied at the seal, and placed in a fume hood to wait for the precipitation of crystals.

3) After the crystals were precipitated, an inverted microscope was used to observe the state of crystal growth and recorded. The crystals were separated from the solution by filtration, and the collected crystals were dried in a dry environment at T=30° C., and a portion of the resulting crystals were sent for XPRD measurement

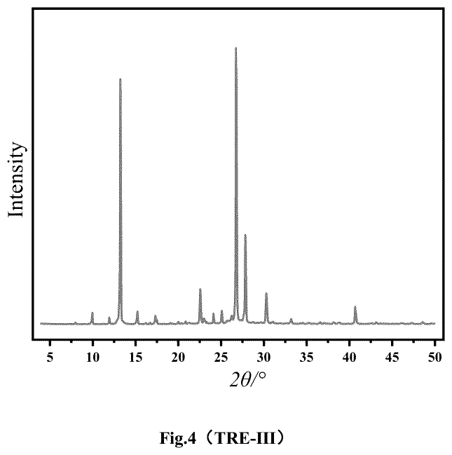

X-ray powder diffraction patterns of crystalline trelagliptin were recorded under the following conditions: room temperature 25° C., relative humidity <60%; XPert PRO-type polycrystalline X-ray diffractometer (PANalytical, The Netherlands), Cu Kα radiation (λ=1.5406 Å), phototube voltage 40 kV, tube current 40 mA, 2θ scanning range 4˜50°, step size 0.01313°, counting time 30 ms/step, and the detection results are shown in FIG. 4. The 2θ values of the new crystalline TRE-II are 8.07°±0.2°, 13.31°±0.2°, 13.64°±0.2°, 16.29°±0.2°, 16.79°±0.2°, 17.40°±0.2°, 19.40°±0.2°, 21.33°±0.2°, 22.83°±0.2°, 26.26°±0.2°, 26.57°±0.2°, 26.79°±0.2°, 27.49°±0.2°, 27.90°±0.2°, 33.33°±0.2°, compared with the existing crystalline forms, there are differences determined as new crystalline type.

### Example 7

Precisely weigh 1 portion of 100 mg of trelagliptin into a 100 mL conical flask, and then add 4 mL of acetone to the conical flask at 25-50° C. until the trelagliptin is dissolved and saturated, and set aside for use.

Take 10 mg of tyrosine (TYR) and add it to the conical flask, add 3 mL of 20% hydrochloric acid to dissolve it until it is saturated.

Then use a rubber-tipped buret to slowly drop the amino acid solution prepared in step 2) into the solution prepared in step 1), the whole process was carried out at 50° C. in a constant temperature water bath;

After the addition was completed, the solution was sealed with plastic wrap and 30-35 apertures were tied at the seal, and placed in a fume hood to wait for the precipitation of crystals.

After the crystals were precipitated, an inverted microscope was used to observe the state of crystal growth and recorded. The crystals were separated from the solution by filtration, and the collected crystals were dried in a dry environment at T=30° C., and a portion of the resulting crystals were sent for XPRD measurement.

X-ray powder diffraction patterns of crystalline trelagliptin were recorded under the following conditions: room temperature 25° C., relative humidity <60%; XPert PRO-type polycrystalline X-ray diffractometer (PANalytical, The Netherlands), Cu Kα radiation (λ=1.5406 Å), phototube voltage 40 kV, tube current 40 mA, 2θ scanning range 4˜50°, step size 0.01313°, counting time 30 ms/step, and the detection results are shown in FIG. 6. The 2θ values of the new crystalline TRE-III are 9.99°±0.2°, 13.29°±0.2°, 15.27°±0.2°, 22.62°±0.2°, 26.79°±0.2°, 27.90°±0.2°, 30.36°±0.2°, 40.73°±0.2°, compared with the existing crystalline forms, there are differences determined as new crystalline type.

### Example 8

Precisely weigh 100 mg of trelagliptin and add it to a 100 mL conical flask, then add 5.5 mL of isopropanol solvent to the conical flask and add it slowly dropwise until the trelagliptin is just dissolved and saturated, and set aside for use.

Then take 4.9 mg of arginine or 4.1 mg of glutamic acid or 4.9 mg of arginine mixed with 4.1 mg of glutamic acid system, either one of which was added to the conical flask, and 3 mL of deionized water was added to dissolve it until it was just dissolved and saturated.

The amino acid solution prepared in step 2) was then slowly dripped into the solution prepared in step 1) sequentially with a rubber-tipped buret, the whole process being carried out in a constant temperature water bath at 40° C.

After the addition was completed, the solution was sealed with plastic wrap and 30-35 apertures were tied at the seal, and placed in a fume hood to wait for the precipitation of crystals.

After the crystals were precipitated, an inverted microscope was used to observe the state of crystal growth and recorded. The crystals were separated from the solution by filtration, and the collected crystals were dried in a dry environment at T=30° C., and a portion of the resulting crystals were sent for XPRD measurement.

X-ray powder diffraction patterns of crystalline trelagliptin were recorded under the following conditions: room temperature 25° C., relative humidity <60%; XPert PRO-type polycrystalline X-ray diffractometer (PANalytical, The Netherlands), Cu Kα radiation (λ=1.5406 Å), phototube voltage 40 kV, tube current 40 mA, 2θ scanning range 4˜50°, step size 0.01313°, counting time 30 ms/step, and the detection results are shown in FIG. 7. The 2θ values of the new crystalline TRE-V are 5.49°±0.2°, 11.04°±0.2°, 12.47°±0.2°, 12.86°±0.2°, 16.59°±0.2°, 20.06°±0.2°, 20.88°±0.2°, 22.19°±0.2°, 28.08°±0.2°, 28.98°±0.2°, 30.47°±0.2°, 33.54±0.2°, compared with the existing crystalline forms, the presence of differences identified as new crystalline forms.

### Example 9

Precisely weigh 200 mg of trelagliptin into a 100 mL conical flask, then add 5 mL of ethanol solvent to the conical flask, slowly titrate until the trelagliptin is just saturated with dissolution, and set aside for use.

Take 20 mg of tyrosine (Tyr) in a conical flask and add 6 mL of 20% hydrochloric acid solution to dissolve until just saturated.

Using a rubber-tipped burette, slowly drop the amino acid solution prepared in 2) into the solution prepared in 1) in sequence, the whole process was carried out in a constant temperature water bath at 37° C.

After the addition was completed, the solution was sealed with plastic wrap and 30-35 apertures were tied at the seal and placed in a fume hood to wait for the precipitation of crystals.

After the crystals were precipitated, an inverted microscope was used to observe the state of crystal growth and recorded. The crystals were separated from the solution by filtration, and the collected crystals were dried in a dry environment at T=30° C., and a portion of the resulting crystals were sent for XPRD measurement.

X-ray powder diffraction patterns of crystalline trelagliptin were recorded under the following conditions: room temperature 25° C., relative humidity <60%; XPert PRO-type polycrystalline X-ray diffractometer (PANalytical, The Netherlands), Cu Kα radiation (λ=1.5406 Å), phototube voltage 40 kV, tube current 40 mA, 2θ scanning range 4˜50°, step size 0.01313°, counting time 30 ms/step, and the detection results are shown in FIG. 4. The 2θ values of the new crystalline TRE-II are 8.07°±0.2°, 13.31°±0.2°, 13.64°±0.2°, 16.29°±0.2°, 16.79°±0.2°, 17.40°±0.2°, 19.40°±0.2°, 21.33°±0.2°, 22.83°±0.2°, 26.26°±0.2°, 26.57°±0.2°, 26.79°±0.2°, 27.49°±0.2°, 27.90°±0.2°, 33.33°±0.2°, compared with the existing crystalline forms, the presence of differences identified as new crystalline forms.

### Example 10

Precisely weigh 100 mg of trelagliptin and add it to a 100 mL conical flask, then add 4.5 mL of isopropanol solvent to the conical flask and add it slowly dropwise until the trelagliptin is just dissolved and saturated, and set aside for use.

Then take 10 mg of arginine or 8 mg of glutamic acid or a mixed system of 10 mg of arginine and 8 mg of glutamic acid, either of which was added to the conical flask, and 2 mL of deionized water was added to dissolve until just saturated with dissolution.

The amino acid solution prepared in step 2) was then slowly dripped into the solution prepared in step 1) sequentially with a rubber-tipped buret, the whole process being carried out in a constant temperature water bath at 45° C.

After the addition was completed, the solution was sealed with plastic wrap and 30-35 apertures were tied at the seal, and placed in a fume hood to wait for the precipitation of crystals.

After the crystals were precipitated, an inverted microscope was used to observe the state of crystal growth and recorded. The crystals were separated from the solution by filtration, and the collected crystals were dried in a dry environment at T=30° C., and a portion of the resulting crystals were sent for XPRD measurement.

X-ray powder diffraction patterns of crystalline trelagliptin were recorded under the following conditions: room temperature 25° C., relative humidity <60%; XPert PRO type polycrystalline X-ray diffractometer (PANalytical, The Netherlands), Cu Kα radiation (λ=1.5406 Å), optical tube voltage 40 kV, tube current 40 mA, 2θ scanning range 4˜50°, step size 0.01313°, counting time 30 ms/step, and the detection results are shown in FIG. 7, and the 2θ values of the new crystalline TRE-V are 5.49°±0.2°, 11.04°±0.2°, 12.47°±0.2°, 12.86°±0.2°, 16.59°±0.2°, 20.06°±0.2°, 20.88°±0.2°, 22.19°±0.2°, 28.08°±0.2°, 28.98°±0.2°, 30.47°±0.2°, 33.54±0.2°, compared with the existing crystalline forms, the presence of differences identified as new crystalline forms.

### Example 11

Precisely weigh 100 mg of trelagliptin and add it to a 100 mL conical flask, then add 5.5 mL of ethyl acetate solvent to the conical flask and add it slowly dropwise until the trelagliptin is just dissolved and saturated, and set aside for use.

Then take 10 mg of arginine or 8 mg of glutamic acid or a mixed system of 10 mg of arginine and 8 mg of glutamic acid, either of which was added to the conical flask, and 2 mL of deionized water was added to dissolve until just dissolved and saturated.

Then use a rubber-tipped buret to slowly drop the amino acid solution prepared in step 2) into the solution prepared in step 1) in sequence, the whole process was carried out in a constant temperature water bath at 50° C.

After the addition was completed, the solution was sealed with plastic wrap and 30-35 apertures were tied at the sealing area, and placed in a fume hood to wait for the precipitation of crystals.

After the crystals were precipitated, an inverted microscope was used to observe the growth state of the crystals and recorded. The crystals were separated from the solution by filtration, and the collected crystals were dried in a dry environment at T=30° C., and a portion of the resulting crystals were sent for XPRD measurement.

X-ray powder diffraction patterns of crystalline trelagliptin were recorded under the following conditions: room temperature 25° C., relative humidity <60%; XPert PRO type polycrystalline X-ray diffractometer (PANalytical, The Netherlands), Cu Kα radiation (λ=1.5406 Å), optical tube voltage 40 kV, tube current 40 mA, 2θ scanning range 4˜50°, step size 0.01313°, counting time 30 ms/step, and the detection results are shown in FIG. 6. The 2θ values of the new crystalline TRE-IV are 5.47°±0.2°, 11.02°±0.2°, 12.88°±0.2°, 16.59°±0.2°, 20.08°±0.2°, 20.91°±0.2°, 22.17°±0.2°, 23.21°±0.2°, 27.84°±0.2°, 33.55°±0.2°, compared with the existing crystalline forms, the presence of differences identified as new crystalline forms.

### Example 12

Precisely weigh 1 portion of 250 mg of trelagliptin into a 100 mL conical flask, then add 6 mL of acetonitrile to the conical flask at 25° C.-50° C. until the trelagliptin is just dissolved and saturated, and set aside for use.

Take another 12.8 mg of tyrosine (TYR) and add it to the conical flask, add 6.5 mL of 20% hydrochloric acid to dissolve it just to saturation.

Then use a rubber-tipped buret to slowly drop the amino acid solution prepared in step 2) into the solution prepared in step 1) sequentially, the whole process was carried out at 50° C. in a constant temperature water bath.

After the addition was completed, the solution was sealed with plastic wrap and 30-35 apertures were tied at the seal, and placed in a fume hood to wait for the precipitation of crystals.

After the crystals were precipitated, an inverted microscope was used to observe the growth state of the crystals and recorded. The crystals were separated from the solution by filtration, and the collected crystals were dried in a dry environment at T=30° C., and a portion of the resulting crystals were sent for XPRD measurement.

X-ray powder diffraction patterns of crystalline trelagliptin were recorded under the following conditions: room temperature 25° C., relative humidity <60%; XPert PRO type polycrystalline X-ray diffractometer (PANalytical, The Netherlands), Cu Kα radiation (λ=1.5406 Å), optical tube voltage 40 kV, tube current 40 mA, 2θ scanning range 4˜50°, step size 0.01313°, counting time 30 ms/step, and the detection results are shown in FIG. 6. The 2θ values of the new crystalline TRE-III are 9.99°±0.2°, 13.29°±0.2°, 15.27°±0.2°, 22.62°±0.2°, 26.79°±0.2°, 27.90°±0.2°, 30.36°±0.2°, 40.73°±0.2°, compared with the existing crystalline forms, the presence of differences identified as new crystalline forms.

In order to prove the effect of the five new crystalline forms of trelagliptin provided in the present application, the solubility of the five new crystalline forms and the original crystalline form in pH=1.2 hydrochloric acid solution, pH=7 aqueous solution, pH=6.8, and pH=7.4 PBS solution was determined below.

The specific operation was as follows: take samples of five new and original crystalline forms after reaching the dissolution equilibrium, filter them with 0.22 μm filter membrane, take appropriate amount of filtrate, according to the UV-Vis spectrophotometric method (Chinese Pharmacopoeia 2020 edition), determine the absorbance value at the wavelength of 279 nm, and substitute them into the standard curve to obtain the solubility of five new and original crystalline forms, as shown in Table 1.

TABLE 1
Solubility table

| | solubility (mg/mL) | | | |
|---|---|---|---|---|
| | | | | pH = 1.2 |
| | | | | hydrochloric |
| Crystal type | pH = 7.0 | pH = 6.8 PBS | pH = 7.4 PBS | acid |
| original | 3.40 | 15.46 | 12.94 | 42.67 |
| crystal form | | | | |
| TRE-I | 13.17 | 34.22 | 28.60 | 54.00 |
| TRE-II | 15.98 | 40.01 | 37.32 | 45.91 |
| TRE-III | 14.93 | 35.90 | 28.26 | 39.76 |
| TRE-IV | 19.56 | 35.05 | 35.94 | 73.49 |
| TRE-V | 12.64 | 36.12 | 37.51 | 76.01 |

The results in Table 1 indicated that the solubility of the five new crystalline forms of trelagliptin found was significantly higher than that of the original crystalline forms in hydrochloric acid solution at pH=1.2, deionized water (pH=7.0), sodium phosphate buffer solution at pH=6.8, and sodium phosphate buffer solution at pH=7.4. The new crystalline forms with high solubility can make up for the defect of poor solubility of the original crystalline forms of trelagliptin.

The foregoing is only a specific embodiment of the present disclosure, not all of the embodiments, and any equivalent transformations adopted by other persons skilled in the art based on the specification of the present disclosure are covered by the claims of the present disclosure and are also protected by the present disclosure.

What is claimed is:

1. A method for preparing new crystal forms of trelagliptin induced by key amino acid based on structure mining of protein-drug interactions, comprising:
introducing an amino acid in a solvent crystallization system of trelagliptin to induce production of five new crystal forms of trelagliptin.

2. The method according to claim 1, comprising the following steps in sequence:
1) slowly adding a solvent dropwise to the trelagliptin until the trelagliptin is just dissolved and saturated to obtain a trelagliptin solution for spare use;
2) adding the amino acid to deionized water or 20 wt % hydrochloric acid solution until the amino acid is dissolved and saturated to obtain an amino acid solution;
3) using a rubber dropper to slowly drop the amino acid solution prepared in step 2) into the trelagliptin solution prepared in step 1) in sequence, wherein the whole process is carried out in a constant temperature water bath at 25° C.-50° C.; and
4) sealing a result product of step 3) with plastic wrap and tying 20-35 apertures at the seal, and placing in a fume hood to wait for precipitation of crystals, to obtain a variety of new crystalline forms of trelagliptin;
wherein a ratio of an amount of matter of the amino acid and the trelagliptin is 1:2˜1:30.

3. The method according to claim 1, wherein the amino acid is one or more selected from a group consisting of glutamic acid, arginine, threonine, and tyrosine; the solvent for dissolving trelagliptin is one of ethyl acetate, isopropanol, acetone, methanol, ethanol, and acetonitrile.

4. new crystal forms of trelagliptin induced by key amino acid based on the structure mining of protein-drug interaction in the method according to claim 1, wherein the new crystalline forms of trelagliptin are TRE-I, TRE-II, TRE-III, TRE-IV, and TRE-V.

5. The new crystalline forms of trelagliptin according to claim 4, wherein the new crystalline form of TRE-I is characterized by an X-ray powder diffraction (XRPD) pattern and has 2θ values at 5.66°±0.2°, 11.35°±0.2°, 12.47°±0.2°, 17.08°±0.2°, 19.88°±0.2°, 22.48°±0.2°, 22.87°±0.2°, 27.54°±0.2°.

6. The new crystalline forms according to claim 4, wherein the new crystalline form of TRE-II is characterized by an XRPD pattern and has 2θ values at 2θ values of 8.07°±0.2°, 13.31°±0.2°, 13.64°±0.2°, 16.29°±0.2°, 16.79°±0.2°, 17.40°±0.2°, 19.40°±0.2°, 21.33°±0.2°, 22.83°±0.2°, 26.26°±0.2°, 26.57°±0.2°, 26.79°±0.2°, 27.49°±0.2°, 27.90°±0.2°, 33.33°±0.2°.

7. The new crystalline forms of trelagliptin according to claim 4, wherein the new crystalline form of TRE-III is characterized by an XRPD pattern and has 2θ values at 9.99°±0.2°, 13.29°±0.2°, 15.27°±0.2°, 22.62°±0.2°, 26.79°±0.2°, 27.90°±0.2°, 30.36°±0.2°, 40.73°±0.2°.

8. The new crystalline forms of trelagliptin according to claim 4, wherein the new crystalline form TRE-IV is characterized by an XRPD pattern and has 2θ values at 5.47°±0.2°, 11.02°±0.2°, 12.88°±0.2°, 16.59°±0.2°, 20.08°±0.2°, 20.91°±0.2°, 22.17°±0.2°, 23.21°±0.2°, 27.84°±0.2°, 33.55°±0.2°.

9. The new crystalline forms of trelagliptin according to claim 4, wherein the new crystalline form TRE-V is characterized by an XRPD pattern and has 2θ values at 5.49°±0.2°, 11.04°±0.2°, 12.47°±0.2°, 12.86°±0.2°, 16.59°±0.2°, 20.06°±0.2°, 20.88°±0.2°, 22.19°±0.2°, 28.08°±0.2°, 28.98°±0.2°, 30.47°±0.2°, 33.54±0.2°.
